(19) United States
(12) Patent Application Publication
(10) Pub. No.: US 2013/0158390 A1
(43) Pub. Date: Jun. 20, 2013

(54) ULTRASONIC NEEDLE GUIDING APPARATUS, METHOD AND SYSTEM

(71) Applicant: General Electric Company, Schenectady, NY (US)

(72) Inventors: Wei Tan, Shanghai (CN); Gang Cheng, Shanghai (CN)

(73) Assignee: General Electric Company, Schenectady, NY (US)

(21) Appl. No.: 13/721,418

(22) Filed: Dec. 20, 2012

(30) Foreign Application Priority Data
Dec. 20, 2011 (CN) 201110429443.9

(51) Int. Cl. A61B 8/08; A61B 10/02; A61B 8/00; A61B 8/14

(52) U.S. Cl. CPC A61B 8/0841; A61B 8/14; A61B 10/0233; A61B 8/4444; A61B 8/5207; A61B 8/461; A61B 8/54
USPC 600/424

(57) ABSTRACT

An apparatus is provided. The apparatus comprises a vibrator configured to vibrate a needle, an ultrasonic scanhead configured to transmit ultrasonic pulses and to receive return signals, and an ultrasonic system coupled to the ultrasonic scanhead. The ultrasonic system comprises a transmitter and receiver module coupled to the ultrasonic scanhead, a displacement estimation module coupled to the transmitter and receiver module, and a display coupled to the displacement estimation module. The transmitter and receiver module is configured to supply energizing pulses to the ultrasonic scanhead to transmit the ultrasonic pulses and to receive electrical signals produced by the ultrasonic scanhead according to the return signals. The displacement estimation module is configured to calculate motion displacements based on phase differences of the electrical signals. The display is configured to display an image according to the motion displacements.

### BACKGROUND OF THE INVENTION

Surgical biopsy procedures or anesthesia procedures are commonly performed with the assistance of ultrasonic imaging to enable the physician to view body tissue. It is desirable during such procedures to be able to clearly visualize the needle and monitor its progression through the body tissue. To enable the physician to view the needle, a conventional needle is vibrated by a vibrator coupled to the needle and a system detects the speed of the needle via a Doppler method and displays an image of the needle. However, in the traditional Doppler method in medical applications, it is difficult to distinguish different frequencies of motion, and it is difficult to track high velocity motion. Therefore, the resolution of the needle in an image through the Doppler method is low.

### BRIEF DESCRIPTION OF THE INVENTION

According to an embodiment disclosed herein, an apparatus is provided. The apparatus comprises a vibrator configured to vibrate a needle, an ultrasonic scanhead configured to transmit ultrasonic pulses and to receive return signals, and an ultrasonic system coupled to the ultrasonic scanhead. The ultrasonic system comprises a transmitter and receiver module coupled to the ultrasonic scanhead, a displacement estimation module coupled to the transmitter and receiver module, and a display coupled to the displacement estimation module. The transmitter and receiver module is configured to supply energizing pulses to the ultrasonic scanhead to transmit the ultrasonic pulses and to receive electrical signals produced by the ultrasonic scanhead according to the return signals. The displacement estimation module is configured to calculate motion displacements based on phase differences of the electrical signals. The display is configured to display an image according to the motion displacements.

According to another embodiment of the present invention, a method is provided. The method comprises vibrating a needle, controlling an ultrasonic scanhead to transmit ultrasonic pulses and to receive return signals, calculating motion displacements based on phase differences of electrical signals which are produced by the ultrasonic scanhead according to the return signals, the motion displacements comprising motion displacements of the needle, and displaying an image of the needle according to the motion displacements of the needle.

According to another embodiment of the present invention, an ultrasonic system is provided. The ultrasonic system comprises a transmitter and receiver module configured to supply energizing pulses and to receive electrical signals, a displacement estimation module coupled to the transmitter and receiver module configured to calculate motion displacements based on phase differences of the electrical signals, and a display coupled to the displacement estimation module configured to display an image according to the motion displacements.

### BRIEF DESCRIPTION OF THE DRAWINGS

These and other features and aspects of embodiments of the present disclosure will become better understood when the following detailed description is read with reference to the accompanying drawings in which like characters represent like parts throughout the drawings, wherein:

FIG. 1 is a schematic block diagram of an ultrasonic needle guiding apparatus in accordance with an exemplary embodiment; and

FIG. 2 is a vibrating wave of the needle used for the ultrasonic needle guiding apparatus shown in FIG. 1.

### DETAILED DESCRIPTION OF THE INVENTION

Unless defined otherwise, technical and scientific terms used herein have the same meaning as is commonly understood by one of ordinary skill in the art to which this disclosure belongs. The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced items. The use of “including,” “comprising” or “having” and variations thereof herein are meant to encompass the items listed thereafter and equivalents thereof as well as additional items. The terms “connected” and “coupled” are not restricted to physical or mechanical connections or couplings, and can include electrical connections or couplings, whether direct or indirect.

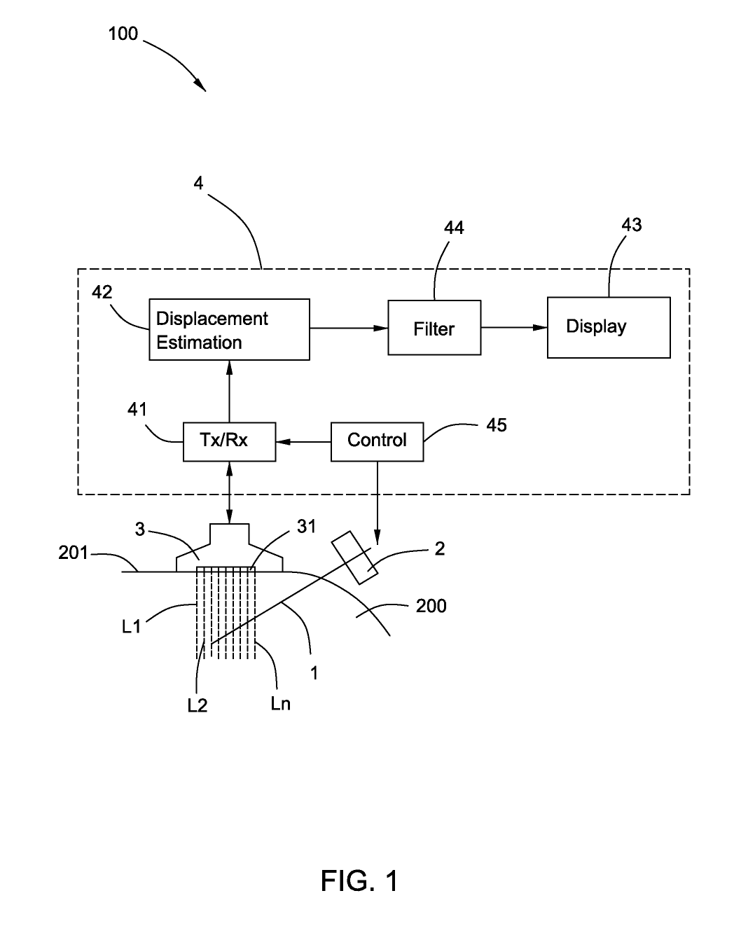

Referring to FIG. 1, an ultrasonic needle guiding apparatus 100 comprises a vibrator 2, an ultrasonic scanhead 3, and an ultrasonic system 4 coupled to the ultrasonic scanhead 3. A needle 1 is configured to be inserted into body tissue 200 in surgical biopsy procedures, anesthesia procedures and other procedures. The needle 1 is coupled to the vibrator 2 before being inserted into the body tissue 200.

The vibrator 2 is configured to vibrate the needle 1. The body tissue 200 close to the needle 1 also vibrates due to the vibration of the needle 1. The vibrator 2 is reciprocated at a frequency which is between 200 Hertz and 2000 Hertz, for example, and the needle 1 is reciprocated at same frequency as the vibrator 2. The vibrator 2 can be reciprocated in any suitable frequency. The frequency of the vibrator 2 and the needle 1 is different from the frequencies of other motions, such as heartbeat, breathing and so on. A vibrating wave of the vibrator 2 and the needle 1 is a sine wave, pulse, sawtooth or other waveform. The vibrator 2 vibrates the needle in a longitudinal direction with respect to the needle 1 or in horizontal direction with respect to the needle 1. The vibrator 2 may be any one of a variety of devices which generate linear reciprocating motion, such as a linear motor, solenoid, speaker coil, or other device capable of developing and coupling longitudinal or/and horizontal reciprocating motion to the needle 1.

The ultrasonic scanhead 3 including imaging transducers 31 is in contact with a surface 201 of skin of a patient for transmitting ultrasonic pulses to the body tissue 200 and receiving return signals from the body tissue 200. The imaging transducers 31 of the ultrasonic scanhead 3 are energized to generate phased array ultrasonic pulses under control of the ultrasonic system 4. The ultrasonic scanhead 3 converts the return signals to electrical signals supplied to the ultrasonic system 4.

The ultrasonic system 4 includes a transmitter and receiver module 41 coupled to the ultrasonic scanhead 3, a displacement estimation module 42 coupled to the transmitter and receiver module 41, and a display 43 coupled to the displacement estimation module 42. The transmitter and receiver module 41 is configured to supply energizing pulses to the ultrasonic scanhead 3 to transmit the ultrasonic pulses and receive the electrical signals produced by the ultrasonic scanhead 3 according to the return signals. The electrical signals are supplied to the displacement estimation module 42.

The displacement estimation module 42 is configured to calculate motion displacements, d, based on phase differences, Δφ, of the electrical signals from the following equations (1):

$$d = \frac{ΔΦ}{2 π} \cdot λ = \frac{ΔΦ}{2 π} \cdot \frac{V}{f} \qquad ( 1 )$$

where λ is a wavelength of the electrical signal, V is a speed of the electrical signal, and f is a frequency of the electrical signal. The motion displacements include motion displacements of the needle 1, vibrating body tissue 200 close to the needle 1 and other motions or movements due to heartbeat, breathing and so on. The motion displacement of the needle 1 is greater than that of other motions.

The display 43 is configured to display an image of the body tissue 200 with the needle 1 therein. The display 43 displays an image of the needle 1 in the body tissue 200 according to the motion displacement of the needle 1. The image of the needle 1 is brighter than the image of the body tissue 200, so the needle 1 can be seen from the display 43. The needle 1 can be displayed on the display 43 in color so as to be easily seen.

In some embodiments, the ultrasonic system 4 further includes a band-pass filter 44 between the displacement estimation module 42 and the display 43 to reject motion displacements which are not motion displacements of the needle 1. The frequency of the vibrator 2 is in a pass band of the band-pass filter 44 so that the motion displacements of the needle 1 remain. The pass band is between 200 Hertz and 2000 Hertz. The pass band can be any suitable value range according to the frequency of the vibrator 2. A bandwidth of the band-pass filter 44 is as narrow as possible in the case of the frequency of the vibrator 2 in the pass band.

A final displacement signal output from the displacement estimation module 42 is supplied to the band-pass filter 44. The final displacement signal is a wave of the motion displacement with respect to time and produced by superimposing displacement signals of the needle 1, vibrating body tissue 200 and other motions. That is to say, an amplitude of the final displacement signal is a sum of the displacements of the needle 1, vibrating body tissue 200 and displacement due to other motions or movement due to heartbeat, breathing and so on. The frequency of the needle 1 is different from frequencies of vibrating body tissue 200 and other motions, so the band-pass filter 44 can reject displacement signals of vibrating body tissue 200 and other motions, respectively. The displacement signal of the needle 1 is supplied to the display 43 and the display 43 displays the image of the needle 1 on the body tissue 200 according the displacement signal of the needle 1. Thereby, it easy to distinguish the needle 1 and the body tissue 200 since interference signals are rejected.

In an embodiment, the ultrasonic pulses from the ultrasonic scanhead 3 are asynchronous with respect to the vibrating wave of the vibrator 2. The energizing pulses from the ultrasonic system 4 are also asynchronous with respect to the vibrating wave of the vibrator 2. A series of the ultrasonic pulses transmitted in a signal direction is referred to as a ray line, as indicated by ray lines L1, L2, . . . Ln in FIG. 1, where n is the number of the ray lines. The ray lines L1, L2, . . . Ln are transmitted line by line. The return signals from the series of the ultrasonic pulses are received with respect to positions along the ray line. A sampling frequency of the return signals is greater than double the frequency of the vibrating wave of the vibrator 2. The sampling frequency can be selected according to the particular application.

In another embodiment, the ultrasonic pulses from the ultrasonic scanhead 3 are synchronized with the vibrating wave of the vibrator 2. The energizing pulses from the ultrasonic system 4 are also synchronized with the vibrating wave of the vibrator 2. The ultrasonic system 4 further includes a controller 45 coupled to the vibrator 2 and the transmitter and receiver module 41 for controlling the vibrator 2 and the transmitter and receiver module 41. The controller 45 controls the transmitter and receiver module 41 to send the energizing pulses synchronized with the motion of the vibrator 2.

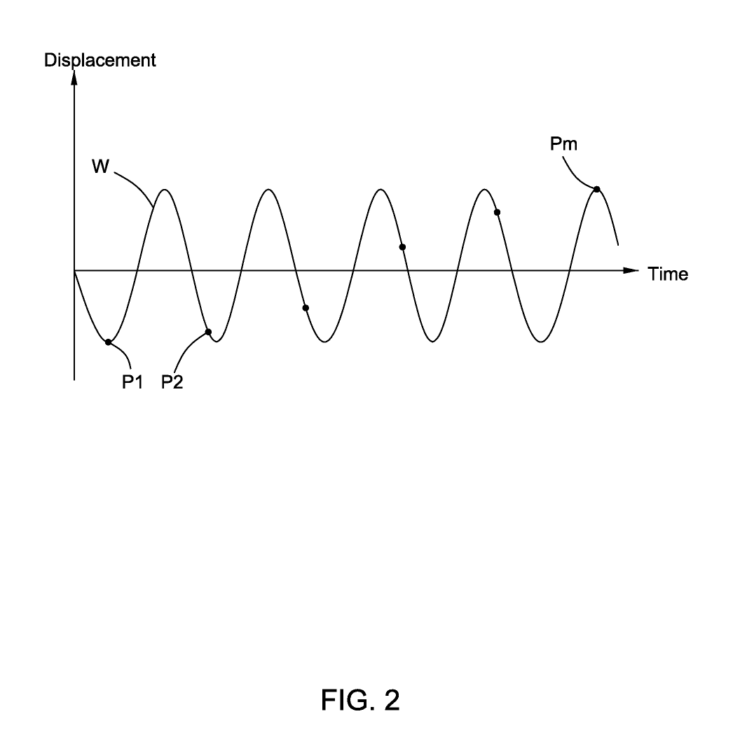

In this embodiment, the ultrasonic scanhead 3 transmits one ultrasonic pulse along one of the ray lines such as L1 and then transmits another ultrasonic pulse along the next ray line such as L2, and so on. The ultrasonic pulses are transmitted one by one along L1 to Ln, called a frame. The ultrasonic scanhead 3 sends the ultrasonic pulses frame by frame. In one frame, a number of different points along the needle 1 are scanned and the return signals from the points are received. Referring to FIG. 2, the vibrating wave W of the needle 1 is a sine wave. The vibrating wave W is sampled by the ultrasonic scanhead 3 via down sampling. Therefore, the frequency of the ultrasonic pulses is low. There are one or two sampling points in one period of the sine wave, as indicated by sampling points P1, P2, . . . Pm in FIG. 2, where m is the number of the sampling points. Thereby, deep positions of the body tissue 200 can also be scanned.

A method for guiding the needle 1 into the body tissue 200 is explained with reference to FIG. 1. The ultrasonic scanhead 3 contacts with the surface 201 of the skin. The needle 1 is vibrated via the vibrator 2 and inserted into the body tissue 200. The ultrasonic system 4 controls the ultrasonic scanhead 3 to transmit the ultrasonic pulses and to receive the return signals. The transmitter and receiver module 41 of the ultrasonic system 4 supplies the energizing pulses to the ultrasonic scanhead 3 to transmit the ultrasonic pulses. The return signals from the body tissue 200 are received and converted to the electrical signals by the transmitter and receiver module 41. In one embodiment, the controller 45 of the ultrasonic system 4 controls the transmitter and receiver module 41 to send the energizing pulses synchronized with the motion of the vibrator 2.

The electrical signals are supplied to the displacement estimation module 42 to calculate the motion displacements based on the phase differences of the electrical signals. The display 43 of the ultrasonic system 4 displays the image of the body tissue 200 and the needle 1 according to the motion displacements. In one embodiment, the motion displacements are filtered by the band-pass filter 44 to show the motion displacement of the needle 1 and then the display 43 displays the needle 1 according to the motion displacements of the needle 1. From the image on the display 43, the needle 1 can be clearly visualized, so the progression of the needle 1 through the body tissue 200 can be monitored.

While embodiments of the invention have been described herein, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

Furthermore, the skilled artisan will recognize the interchangeability of various features from different embodiments. The various features described, as well as other known equivalents for each feature, can be mixed and matched by one of ordinary skill in this art to construct additional systems and techniques in accordance with principles of this disclosure.

What is claimed is:

1. An apparatus comprising:
a vibrator configured to vibrate a needle;
an ultrasonic scanhead configured to transmit ultrasonic pulses and receive return signals; and
an ultrasonic system coupled to the ultrasonic scanhead, the ultrasonic system comprising:
a transmitter and receiver module coupled to the ultrasonic scanhead and configured to supply energizing pulses to the ultrasonic scanhead to transmit the ultrasonic pulses, and to receive electrical signals produced by the ultrasonic scanhead according to the return signals;
a displacement estimation module coupled to the transmitter and receiver module configured to calculate motion displacements based on phase differences of the electrical signals; and
a display coupled to the displacement estimation module configured to display an image according to the motion displacements.

2. The apparatus of claim 1, wherein the ultrasonic system further comprises a band-pass filter coupled between the displacement estimation module and the display, wherein the vibrator is reciprocated at a frequency which is in a pass band of the band-pass filter.

3. The apparatus of claim 2, wherein the frequency of the vibrator is between about 200 Hertz and about 2000 Hertz.

4. The apparatus of claim 1, wherein the ultrasonic pulses from the ultrasonic scanhead are asynchronous with a vibrating wave of the vibrator.

5. The apparatus of claim 1, wherein the ultrasonic system further comprises a controller coupled to the vibrator and to the transmitter and receiver module, the control being configured to control the vibrator and the transmitter and receiver module, and wherein the ultrasonic pulses from the ultrasonic scanhead are synchronized with a vibrating wave of the vibrator.

6. The apparatus of claim 5, wherein the vibrating wave is sampled by the ultrasonic scanhead via down sampling.

7. The apparatus of claim 6, wherein the vibrating wave is a sine wave.

8. A method comprising:
vibrating a needle;
controlling an ultrasonic scanhead to transmit ultrasonic pulses and to receive return signals;
calculating motion displacements based on phase differences of electrical signals which are produced by the ultrasonic scanhead according to the return signals, the motion displacements comprising motion displacements of the needle; and
displaying an image of the needle according to the motion displacements of the needle.

9. The method of claim 8, further comprising filtering the motion displacements using a band-pass filter to reject motion displacements which are not motion displacements of the needle.

10. The method of claim 9, wherein the vibrator is reciprocated at a frequency which is in a pass band of the band-pass filter and between about 200 Hertz and about 2000 Hertz.

11. The method of claim 8, wherein the ultrasonic pulses from the ultrasonic scanhead are asynchronous with a vibrating wave of the vibrator.

12. The method of claim 8, wherein the ultrasonic pulses from the ultrasonic scanhead are synchronized with a vibrating wave of the vibrator.

13. The method of claim 12, wherein the vibrating wave is sampled by the ultrasonic scanhead via down sampling.

14. The method of claim 13, wherein the vibrating wave is a sine wave.

15. The method of claim 8, wherein the vibrator vibrates the needle in a longitudinal direction with respect to the needle.

16. The method of claim 8, wherein the vibrator vibrates the needle in a horizontal direction with respect to the needle.

17. An ultrasonic system comprising:
a transmitter and receiver module configured to supply energizing pulses and to receive electrical signals;
a displacement estimation module coupled to the transmitter and receiver module configured to calculate motion displacements based on phase differences of the electrical signals; and
a display coupled to the displacement estimation module configured to display an image according to the motion displacements.

18. The ultrasonic system of claim 17, wherein the ultrasonic system further comprises a band-pass filter between the displacement estimation module and the display.

19. The ultrasonic system of claim 18, wherein a pass band of the band-pass filter is between about 200 Hertz and about 2000 Hertz.

20. The ultrasonic system of claim 17, wherein the ultrasonic system further comprises a controller coupled to the transmitter and receiver module, the controller being configured to control the transmitter and receiver module.
